(19) United States
(12) Patent Application Publication
(10) Pub. No.: US 2026/0108244 A1
(43) Pub. Date: Apr. 23, 2026

(54) SURGICAL ELEMENT HOUSINGS, STRUCTURES, CONTENTS, AND ACCESSORIES

(71) Applicant: Origami Surgical, Inc., Madison, NJ (US)

(72) Inventors: Patrick John Culligan, Madison, NJ (US); John Hironimus, Charlotte, NC (US); Dan Shoemaker, Charlotte, NC (US)

(21) Appl. No.: 18/924,352

(22) Filed: Oct. 23, 2024

(51) Int. Cl. A61B 17/06; A61B 17/04

(52) U.S. Cl. CPC A61B 17/06128; A61B 2017/0479; A61B 2017/06147

(57) ABSTRACT

A number of surgical element carriers, housings, structures, contents, and accessories are described. An example surgical element carrier includes a housing dimensioned for insertion through a surgical incision during a surgery. The housing includes a first housing portion, a second housing portion attached and movable relative to the first housing portion between closed and open positions of the housing, and a cover over the second housing portion. The cover forms a boundary of an interior space within the second housing portion. In one example, the cover includes a plurality of slits that intersect with each other for a passageway through the cover, from outside to within the interior space within the second housing portion.

### BACKGROUND

Many surgeries are performed using robotic technology, such as surgical systems commonly used for laparoscopic surgeries. Surgical element carriers have been designed to include a housing that may be inserted through a surgical port during a surgery that can be manipulated during the surgery. For instance, a clam-shell type of housing of a surgical element carrier may be sized and dimensioned to contain surgical elements therein, such as sutures. Ends of the housing are designed such that they can be manipulated by a surgeon or by a robotic manipulator.

### SUMMARY

A number of surgical element carriers, housings, structures, contents, and accessories are described. An example surgical element carrier includes a housing dimensioned for insertion through a surgical incision during a surgery. The housing includes a first housing portion, a second housing portion attached and movable relative to the first housing portion between closed and open positions of the housing, and a cover over the second housing portion. The cover forms a boundary of an interior space within the second housing portion. In one example, the cover includes a plurality of slits that intersect with each other for a passageway through the cover, from outside to within the interior space within the second housing portion. The housing can include a coned end at one end of the housing and an end tab at another end of the housing.

In some examples, the slits include two intersecting slits that form a plus sign opening through the cover. In other examples, the slits form a star opening through the cover. The slits can include a plurality of intersecting perforations in other examples. The cover can also include a second plurality of slits that intersect with each other for a second passageway through the cover, and additional groups of slits can be relied upon.

In other aspects, the surgical element carrier can also include a foam block secured to a top surface of the cover, a magnetic member secured within the interior space within the second housing portion, and/or a gel that occupies at least a portion of the interior space within the second housing portion. The surgical element carrier can also include one or more surgical elements positioned in the first housing portion.

An example surgical element carrier assembly includes a housing dimensioned for insertion through a surgical incision during a surgery. The housing includes a first housing portion, a second housing portion attached and movable relative to the first housing portion between closed and open positions of the housing, and a cover over the second housing portion. The cover forms a boundary of an interior space within the second housing portion. In one example, the cover includes a plurality of slits that intersect with each other for a passageway through the cover, from outside to within the interior space within the second housing portion. The assembly also includes an endcap cone configured to fit over one end of the housing.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1A is a perspective view of a housing for surgical elements according to various embodiments of the present disclosure.

FIG. 1B is a side view of the housing shown in FIG. 1A according to various embodiments of the present disclosure.

FIG. 1C is a perspective view of the housing shown in FIG. 1A, empty and opened, according to various embodiments of the present disclosure.

FIG. 1D is a perspective view of the open housing shown in FIG. 1C with a cover over a portion of the housing according to various embodiments of the present disclosure.

FIG. 1E is a perspective view of the open housing shown in FIG. 1C with a cover over a portion of the housing and substance under the cover according to various embodiments of the present disclosure.

FIG. 1F is a perspective view of the open housing shown in FIG. 1C with a substance in a portion of the housing according to various embodiments of the present disclosure.

FIG. 1G is a perspective view of the open housing shown in FIG. 1C with a perforated cover over a portion of the housing according to various embodiments of the present disclosure.

FIG. 1H is a side view of the housing shown in FIG. 1G with a magnetic adhesion member in the housing according to various embodiments of the present disclosure.

FIG. 2A is a side view of another housing according to various embodiments of the present disclosure.

FIG. 2B is a side view of another housing according to various embodiments of the present disclosure.

FIG. 3A is a side view of the housing shown in FIG. 1A with an endcap cone according to various embodiments of the present disclosure.

FIG. 3B is a side view of the housing shown in FIG. 3A with the endcap cone over an end of the housing according to various embodiments of the present disclosure.

FIG. 3C is a side view of the housing shown in FIG. 3A with a sectional view of the endcap cone over an end of the housing according to various embodiments of the present disclosure.

FIG. 4 illustrates example endcap cones of various profile shapes according to various embodiments of the present disclosure.

### DETAILED DESCRIPTION

The embodiments described herein are directed to surgical element carriers, housings, contents, and accessories. The carriers can be relied upon for housing surgical elements, such as needles with associated/threaded sutures. Such canisters can be used with robotic surgical technology and can be placed and manipulated inside the body of a human or animal subject. The canisters can also be used in standard laparoscopic surgery such as when a practitioner (e.g., a surgeon) performs an intra-corporeal knot tying technique or other technique for “auto-fixation” of suture material. The embodiments can be used in any robotic-assisted endoscopic surgical procedure that requires the placement of multiple sutures, including gynecologic surgery, urogynecologic surgery, urologic surgery, thoracic surgery, and other surgical procedures.

Each carrier can hold a number of new, sterile needles. The carrier can also serve as a needle disposal device. The disposal area of the carrier provides a safe storage area for used needles and allows the practitioner to safely dispose of used needles. Additionally, by containing the used needles with the disposal area, operating staff and nurses can easily check and account for all used needles after the carrier has been removed from the human or animal subject.

Thus, aspects of surgical element carriers, housings, structures, contents, and accessories are described herein. An example surgical element carrier includes a housing dimensioned for insertion through a surgical incision during a surgery. The housing includes a first housing portion, a second housing portion attached and movable relative to the first housing portion between closed and open positions of the housing, and a cover over the second housing portion. The cover forms a boundary of an interior space within the second housing portion. In one example, the cover includes a plurality of slits that intersect with each other for a passageway through the cover, from outside to within the interior space within the second housing portion.

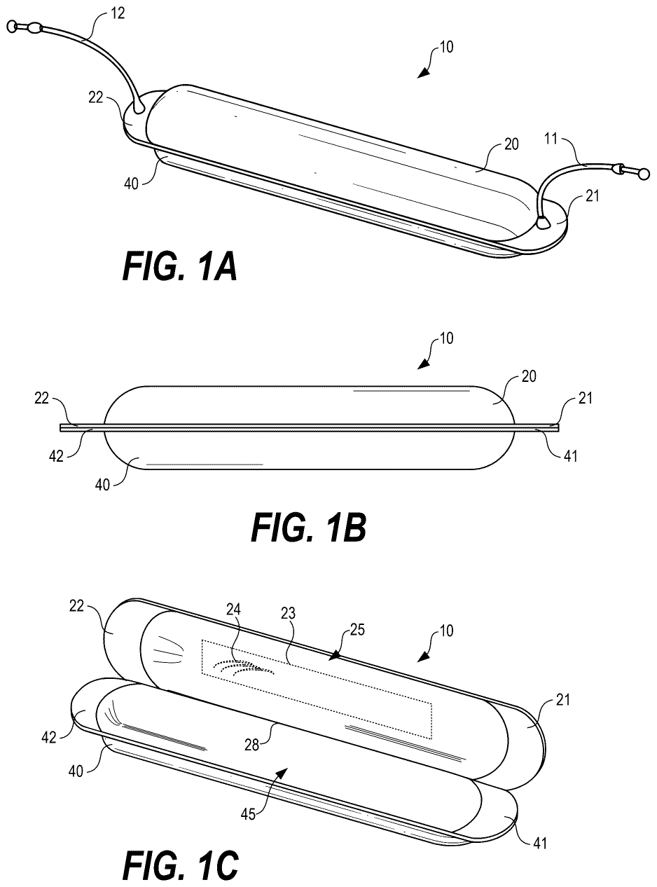

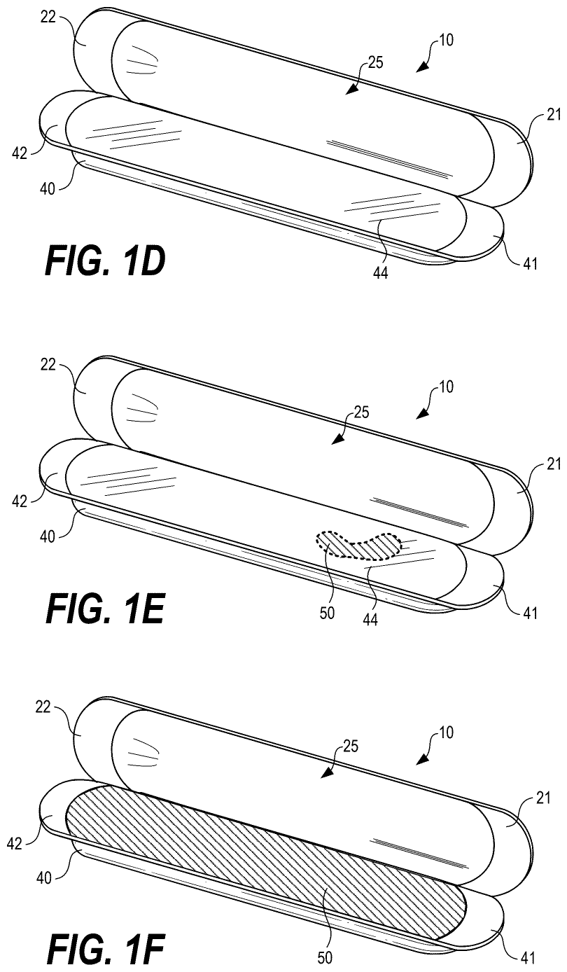

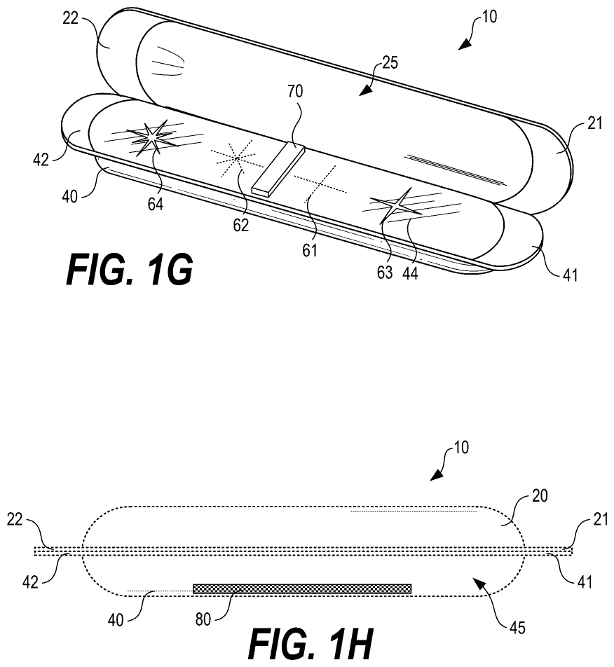

FIG. 1A is a perspective view of a housing 10 for surgical elements according to various embodiments of the present disclosure. FIG. 1B is a side view of the housing 10 shown in FIG. 1A. FIG. 1C is a perspective view of the housing 10 shown in FIG. 1A, empty and opened. The housing 10 is illustrated as a representative example among FIGS. 1A-1C. The housing 10 can vary in shape, size, and materials among the embodiments. In the example shown, the housing 10 is generally cylindrical in shape and formed from a relatively rigid material. Any suitable biocompatible material can be used to form the housing 10, such as a relatively rigid plastic or polymer, including a liquid crystal polymer (LCP), polyethylene (PE), polytetrafluoroethylene (PTFE), fluoropolymer, or other plastic(s) or insulating material(s). The housing 10 can also be formed from other materials, including metals such as stainless steel, and other rigid materials. In other cases, the housing 10 can also be formed from flexible materials, including softer plastics, rubber, or other materials.

The housing 10 includes a first housing portion 20 and a second housing portion 40. The first housing portion 20 is attached to and movable (e.g., rotatable) relative to the second housing portion 40 between closed and open positions. Stated another way, the second housing portion 40 is attached to and movable relative to the first housing portion 20. The first housing portion 20 is attached to the second housing portion 40 along a hinge 28 of the housing 10, which extends along a longitudinal edge of the housing 10 between the housing portions 20 and 40. The housing 10 can be manipulated between open and closed positions. FIGS. 1A and 1B illustrate examples where the housing 10 is closed, and FIG. 1C illustrates an example where the housing 10 is open.

As example dimensions, the housing 10 can be approximately 5 cm long (e.g., measured from right to left of the page in FIG. 1B) and 11 mm in diameter. The housing 10 can have a diameter or largest cross-sectional dimension that is less than about 12 mm in preferred embodiments, which allows the housing 10 to be inserted through a typical surgical incision or trocar for laparoscopic surgery. The housing 10 is not limited to any particular size, however, and the length and the sectional diameter of the housing 10 can range from that described above. Although the housing 10 is depicted as being generally cylindrical in shape, the housing 10 can be formed to other shapes in other cases, and examples of alternative shapes are described below.

The first housing portion 20 includes end tabs 21 and 22, and the second housing portion 40 includes end tabs 41 and 42. The tabs 21, 22, 41, and 42 can be manipulated by hand, by robotic surgical tools, or other approaches to open and close the housing 10. The housing 10 also includes a first tab cord 11 and a second tab cord 12 in some cases, as depicted in FIG. 1A. The first tab cord 11 is attached and/or extends through one or both of the tabs 21 and 41, and the second tab cord 12 is attached and/or extends through one or both of the tabs 22 and 42. The tab cords 11 and 12 can serve a range of purposes, such as a latch mechanism to open and close the housing 10, to keep the housing 10 securely closed during insertion and removal of housing 10 from a subject, for removing the housing 10 from a subject, or for other purposes.

The housing 10 encloses or surrounds an interior space as shown in FIG. 1C. The interior space is provided within the first housing portion 20 and the second housing portion 40. A first interior space 25 is available within the first housing portion 20, and a second interior space 45 is available within the second housing portion 40. A range of different surgical elements and instruments, medicaments, and other items or substances can be positioned and contained within the housing 10. In preferred embodiments, one or more needles and surgical threads (e.g., sutures) can be positioned and secured within the first interior space 25 of the first housing portion 20. The needles and surgical threads can be relied upon to perform a range of different surgical procedures, such as laparoscopic surgeries using robotic manipulators. As one example, a package 23 can be positioned and secured within the first interior space 25. The package 23 is drawn as a representative example in FIG. 1C, as it can vary in shape, form, and composition among the embodiments. The package 23 can include a suture needle 24, and the suture needle 24 can include a surgical thread (not shown) attached to it. The package 23 can include any number of needles and threads, to the extent practical and to fit into the first interior space 25. Additionally, the second interior space 45 within the second housing portion 40 can be relied upon as a type of “sharps” container, in various ways, as described in further detail below.

As other examples, one or more of the surgical elements or instruments described in U.S. Pat. No. 8,418,851 (the '851 patent) can be positioned, contained, and secured in the first interior space 25, and the entire disclosure of the '851 patent is incorporated herein by reference. The apparatus 210 for holding needles 12, the apparatus 310 for holding needles 12, or the other structures for holding needles described in the '851 patent can be positioned, contained, and secured in the first interior space 25. As other examples, one or more of the surgical elements or instruments described in U.S. Patent Publication No. 2022/0346778 (the '778 Publication) can be positioned, contained, and secured in the first interior space 25, and the entire disclosure of the '778 Publication is incorporated herein by reference. As a more particular example, the surgical elements 120 described in the '778 Publication can be positioned, contained, and secured in the first interior space 25. As other examples, any of the biologics described in U.S. Patent Publication No. 2023/0414910 (the '910 Publication) can be positioned, contained, and secured in the first interior space 25, and the entire disclosure of the '910 Publication is incorporated herein by reference.

FIG. 1D is a perspective view of the open housing 10 shown in FIG. 1C with a cover 44 over the second housing portion 40. The cover 44 encloses the second interior space 45 within the second housing portion 40. The cover 44 can be embodied as a relatively thin sheet of material that extends over and encloses the second interior space 45 of the second housing portion 40. The cover 44 can be formed from a range of different materials among the embodiments. As one example, the cover 44 can be formed from the same material as the housing 10, such as a plastic or polymer. The cover 44 can be formed to be the same thickness as the remainder of the second housing portion 40 in one example. However, the cover 44 can also be formed to be thinner than the remainder of the second housing portion 40 in other cases. The cover 44 can also be formed from a material that is different than the housing 10. For example, the second housing portion 40 can be formed from a first type of polymer or plastic, and the cover 44 can be formed from a second, different type of polymer or plastic. Although not illustrated in FIG. 1D, the first housing portion 20 can also include a cover similar to the cover 44, to enclose the first interior space 25 within the first housing portion 20.

In practice, a needle can be used to puncture the cover 44. The needle can puncture the cover 44 and, if desired, be pushed through the cover 44 and into the second interior space 45 of the second housing portion 40, where the needle will be trapped and secured. The second interior space 45 within the second housing portion 40 can be open (i.e., open space or air). The second interior space 45 can be relied upon as a type of “sharps” container based on the coverage provided by the cover 44. In the example depicted in FIG. 1D, because the second interior space 45 is unoccupied and open space, any needles that are inserted through the cover 44 and into the interior space 45 may move, slide, or shake around within the interior space 45. In other cases, the second interior space 45 can be filled or occupied with one or more materials or substances, as described below.

FIG. 1E is a perspective view of the open housing 10 shown in FIG. 1C with the cover 44 over the second housing portion 40 and one or more material(s) or substance(s) 50 (also “substance 50”) under the cover 44. In the example illustrated in FIG. 1E, the substance 50 is sealed and enclosed within the second housing portion 40 under the cover 44. However, as described in further detail below, the cover 44 can be pierced, cut open, or include perforations or openings that facilitate access to the substance 50. The substance 50 can be embodied as a single material or substance or composition of two or more materials or substances. The substance 50 can be relied upon for a range of different purposes. In one example, the substance 50 can be relied upon to envelop and hold (e.g., grip and secure) one or more surgical needles, such as after the needles have been used to stitch one or more sutures, acting as a type of “sharps” container. The substance 50 can be embodied as a foam, rubber, gel, or a related type of semi-solid or elastic, deformable material. Biocompatible materials can be preferrable for the substance 50, such as certain polymer-based foams, hydrogels, organogels, and other types of materials. In other cases, the substance 50 can be embodied as semi-rigid structures, such as corrugated or honeycomb structures including air cavities (e.g., air pockets) distributed among relatively thin vanes of polymer or related materials. Generally, the substance 50 is capable of enveloping and holding one or more surgical needles, either by mechanical friction, adhesion, or a combination of mechanical friction and adhesion.

FIG. 1F is a perspective view of the open housing 10 shown in FIG. 1C with the substance 50 in the second housing portion 40. The example shown in FIG. 1F is similar to that shown in FIG. 1E but omits the cover 44. Thus, a larger surface area of the substance 50 is exposed. Depending on the composition of the substance 50, an adhesive, weld (e.g., plastic weld or melt), or other fastening means can be relied upon in some cases to hold the substance 50 in place within the second housing portion 40. In any case, the substance 50 can be relied upon to envelop and hold (e.g., grip and secure) one or more surgical needles, such as after the needles have been used to stitch one or more sutures, acting as a type of “sharps” container. The exposed top surface of the substance 50 can be planar or substantially planar in some cases. The exposed top surface of the substance 50 can be coplanar with the surfaces of the tabs 41 and 42. In other cases, the exposed top surface of the substance 50 can be sunken down below the surfaces of the tabs 41 and 42 or expanded up (e.g., bulged) and beyond the surfaces of the tabs 41 and 42.

FIG. 1G is a perspective view of the open housing 10 shown in FIG. 1C with the cover 44 including perforations and openings. The cover 44 shown in FIG. 1G is similar to the cover 44 shown in FIGS. 1D and 1E but also includes perforations 61 and 62 and openings 63 and 64. The perforations 61 and 62 can be embodied as one or more rows of small indentations or holes punched in or through the cover 44, so that a slit or opening can be punched through the cover 44 relatively easily. The perforation 61 is formed of two rows of small indentations or holes that extend orthogonal to and intersect with each other in the shape of a plus (+) sign. Each row among the two rows of the perforation 61 have the same length. The perforation 62 is formed of four rows of small indentations or holes that extend in different directions and intersect with each other to form the shape of a star. The rows of the perforation 62 can have the same or different lengths.

The cover 44 can be split open at the locations of the perforations 61 and 62 with sufficient pressure or force applied at or to the perforations 61 and 62. The pressure or force can be applied using even a blunt edge or surface, and the cover 44 can be opened at the locations of the perforations 61 and 62 without a sharp instrument or object. The perforations 61 and 62 facilitate the formation of an opening through the cover 44, at a particular location and limited to a particular size, at the direction of an individual using or manipulating the housing 10. The shapes, sizes, and locations of the perforations 61 and 62 can vary as compared to that shown. Also, either or both of the perforations 61 and 62 can be omitted in some cases, and additional perforations can be included in other cases.

The openings 63 and 64 are examples of the result of breaking through perforations, such as the perforations 61 and 62, respectively. The opening 63 is shaped like a plus, and the opening 64 is shaped like a star. Depending on the type of material from which the cover 44 is formed, the flaps of the openings 63 and 64 can be resilient and elastic. Thus, with applied pressure against the flaps of the openings 63 and 64, the flaps can be pushed down into the second interior space 45 of the second housing portion 40. After the pressure or force is removed, the flaps of the openings 63 and 64 can return to a more co-planar region with the remainder of the top surface of the cover 44, forming a type of closed door. It should be appreciated that the perforations in the cover 44 can take other shapes. The cover 44 can be formed to include single slit perforations, perforations in “U,” “C,” “L,” “V,” “T,” or other shapes. Additionally, the embodiment depicted in FIG. 1G can be combined with that shown in FIG. 1E, such that the substance 50 is positioned under the cover 44 with the perforations.

It is not necessary in all cases for the openings 63 and 64 to be the result of breaking through perforations in the cover 44. Each of the openings 63 and 64 can be formed by two or more slit openings (i.e., rather than perforations that have been broken or opened) through the cover 44. The opening 63 can be formed of two slit openings through the cover 44. The two slit openings extend orthogonal to each other in the shape of a plus (+) sign and intersect with each other. The opening 64 can be formed of four slit openings through the cover 44 that extend in different directions. The four slit openings intersect with each other and form the shape of a star. Each of the slit openings that form the openings 63 and 64 can have the same or different lengths. The openings 63 and 64, among other openings through the cover 44, can be formed from other numbers of slit openings, including 3, 5, 6, 7, 8, or more slit openings. The slit openings can intersect with each other in a variety of ways. Thus, the openings through the cover 44 can be formed in “U,” “C,” “L,” “V,” “T,” and other shapes. As used herein, the term “slit” includes both perforations which can be broken into slit openings and slit openings.

The openings 63 and 64 thus comprise a number of flaps, each of which is triangular in shape. Depending on the type of material from which the cover 44 is formed, the flaps of the openings 63 and 64 can be resilient and elastic. Thus, with applied pressure against the flaps of the openings 63 and 64, the flaps can be pushed down into the second interior space 45 of the second housing portion 40. After the pressure or force is removed, the flaps of the openings 63 and 64 can return to a more co-planar region with the remainder of the top surface of the cover 44, forming a type of closed door. It should be appreciated that the perforations in the cover 44 can take other shapes. The cover 44 can be formed to include single slit perforations, perforations in “U,” “C,” “L,” “V,” “T,” or other shapes. Additionally, the embodiment depicted in FIG. 1G can be combined with that shown in FIG. 1E, such that the substance 50 is positioned under the cover 44 with the perforations.

A foam block 70 is also positioned over the cover 44 in the example shown in FIG. 1G. The foam block 70 can be embodied as a block or strip of polymer-based foam or related material. The foam block 70 can be secured to the top surface of the cover 44 using adhesives, mechanical interferences, or other means. The tip of a needle can be pushed into the foam block 70, to secure the needle in place temporarily. The foam block 70 provides a location for a user of the housing 10 to secure and hold needles before (or during) stitching, for example, before the needles are ultimately secured in the second interior space 45 of the second housing portion 40. A single foam block 70 is shown in FIG. 1G. Two or more foam blocks similar to the foam block 70 can be positioned over the cover 44 in other cases, however, and the foam blocks can be positioned between any of the perforations 61 and 62 and the openings 63 and 64.

FIG. 1H is a side view of the housing 10 shown in FIG. 1G with a magnetic member 80 secured in the second interior space 45 of the housing 10. The magnetic member 80 can be embodied as a permanent magnet, such as a neodymium, cobalt, ferrite, or other magnet. The magnetic member 80 can be formed in a range of different sizes and shapes. The magnetic member 80 can be secured to the inner surface of the second housing portion 40 using adhesives, mechanical fasteners, mechanical interferences, or other suitable means. The magnetic member 80 can attract needles within the second interior space 45 of the housing 10, securing them in place. Thus, once a needle is inserted though the cover 44, it can be attracted to the magnetic member 80 and held in place against it. The cover 44 can even be omitted in some cases when the magnetic member 80 is positioned within the second interior space 45.

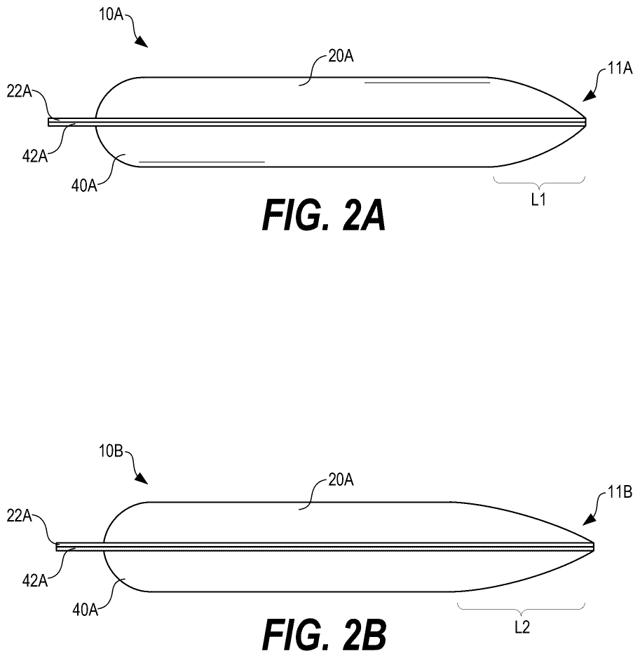

FIG. 2A is a side view of another housing 10A according to various embodiments of the present disclosure. The housing 10A is illustrated as a representative example. The housing 10A can vary in shape, size, and materials among the embodiments. The housing 10A is generally cylindrical in shape, with a coned or beaked end, and formed from a relatively rigid material. Any suitable biocompatible material can be used to form the housing 10A, including those described herein and others. The housing 10A includes a first housing portion 20A and a second housing portion 40A. The first housing portion 20A is attached to and movable (e.g., rotatable) relative to the second housing portion 40A between closed and open positions. The first housing portion 20A is attached to the second housing portion 40A along a hinge of the housing 10A, which extends along a longitudinal edge of the housing 10A between the housing portions 20A and 40A. The housing 10A can be manipulated between open and closed positions, similar to the housing 10 described above.

At one end of the housing 10A, the first housing portion 20A includes an end tab 22A, and the second housing portion 40A includes an end tab 42A. The end tabs 22A and 42A can be manipulated by hand, by robotic surgical tools, or other approaches to open and close the housing 10A. The housing 10A can also include one or more tab chords (not shown) in some cases, similar to those depicted in FIG. 1A. At another end of the housing 10A, the housing 10A includes a coned or beaked end 11A (“coned end 11A”). The coned end 11A can be helpful to insert the housing 10A through a surgical incision, particularly in situations where a trocar is unavailable too thin for insertion of the housing 10A through the trocar. When a surgical incision is made, the skin, facia, and peritoneum can shift to some extent with respect to each other. The coned end 11A can be helpful to navigate the housing 10A through those layers and the surgical incision, due to the coned shape.

The length L1 of the coned end 11A can vary among the embodiments. The length L1 of the coned end 11A can be measured from where the outer cylindrical surface of the housing 10A begins to taper, to the tip end of the housing 10A. Example lengths L1 of the coned end 11A can range from between 1 to 10 cm or more. The coned end will also vary in shape depending on the length of the end. FIG. 2B illustrates an example of the housing 10B, which includes a coned end 11B. The coned end 11B in FIG. 2B is similar to the coned end 11A in FIG. 2A, but the coned end 11B is longer than the coned end 11A. For being longer, the coned end 11B is more tapered and narrowed than the coned end 11A. The length L2 of the coned end 11B can be greater than 3 cm.

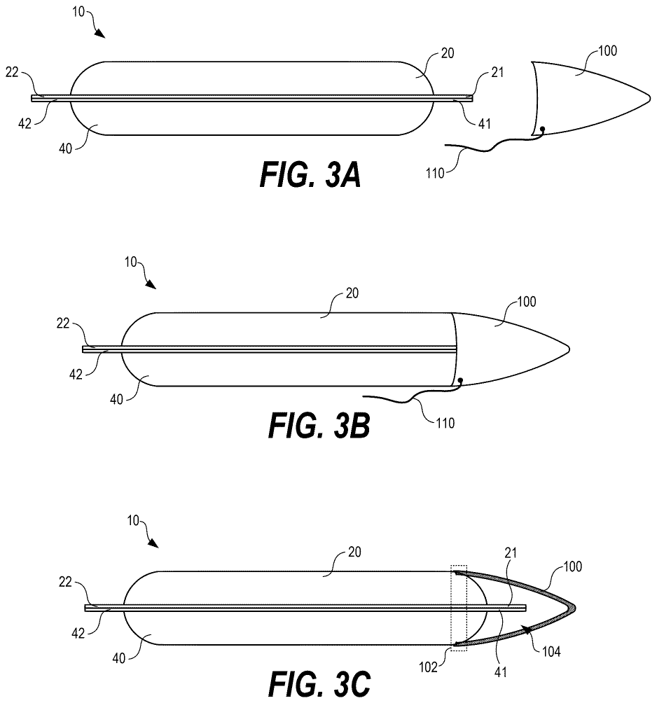

In other cases, the housing 10 can be paired or fitted with a separate endcap cone. FIG. 3A is a side view of the housing 10 shown in FIG. 1A and an endcap cone 100 separate from the housing 10. FIG. 3B is a side view of the housing 10 with the endcap cone 100 over an end of the housing 10, and FIG. 3B is a side view of the housing 10 with a sectional view of the endcap cone 100 over an end of the housing 10. The endcap cone 100 is illustrated as a representative example and can vary in shape, size, and materials among the embodiments. In the example shown, the endcap cone 100 is formed in the shape of a beak, bullet, or cone. The endcap cone 100 can be formed from an LCP, PE, PTFE, fluoropolymer, or other plastic(s) or insulating material(s). The endcap cone 100 can also be formed from other materials, including metals such as stainless steel, and other rigid materials. In other cases, the endcap cone 100 can also be formed from flexible materials, including softer plastic(s), rubber(s), or other materials. The endcap cone 100 also includes an endcap chord 110, which can extend to any suitable length. The endcap chord 110 is attached at one end to the endcap cone 100. The endcap chord 110 can be relied upon to remove the endcap cone 100 from a surgical site, as one example, or for other purposes. The other end of the endcap chord 110, which is shown free in FIG. 3A, can also be affixed to the housing 10 in some cases.

As best shown in FIGS. 3B and 3C, the endcap cone 100 can be fitted over one end of the housing 10. The endcap cone 100 is formed as a type of shell with an open end 102 and free space 104 (FIG. 3C) within the endcap cone 100. The open end 102 of the endcap cone 100 can be fitted over an end of the housing 10, as shown in FIGS. 3B and 3C. The edge of the open end 102 of the endcap cone 100 can be pressed against the outer, cylindrical surface at one end of the housing 10, and the endcap cone 100 can be secured in that position with an interference fit over the housing 10 in one example. The endcap cone 100 can also be made of a material that is relatively flexible and, in some cases, more flexible (e.g., bendable, stretchable, etc.) than the housing 10. Thus, the open end 102 of the endcap cone 100 can expand to some extent in some cases and be pulled over the outer surface of the housing 10. The free space 104 within the endcap cone 100 can also be made to form a partial vacuum in some cases, particularly if the endcap cone 100 is squeezed in part when it is positioned over the end of the housing 10. The partial vacuum can also help to secure the endcap cone 100 over the housing 10.

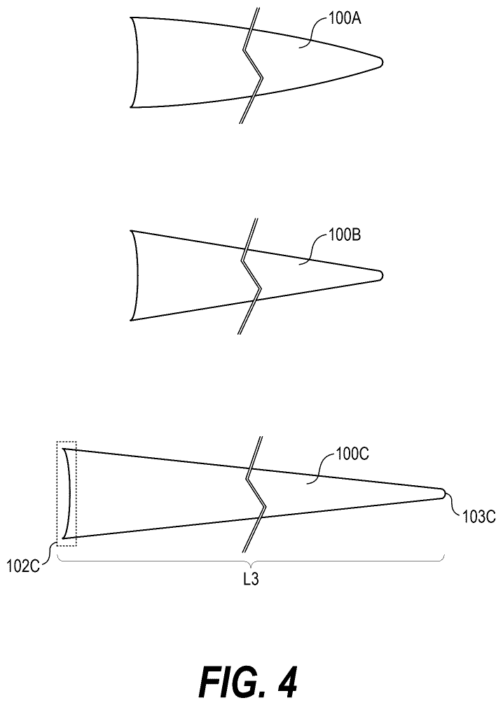

FIG. 4 illustrates other examples of endcap cones. The endcap cone 100A is longer and more narrow than the endcap cone 100. The endcap cone 100B is more pointed than the endcap cone 100A. The endcap cone 100A is ballistic in shape, including curved outer surfaces, while the endcap cone 100B is conical in shape. The endcap cone 100C is longer than the endcap cone 100B. The example endcap cones shown in FIG. 4 can be formed to any suitable length. The length of the endcap cone 100C, which can be measured from the open end 102 to the front tip 103C of the endcap cone 100C, can range from between 1 to 10 cm or more. In other examples, the length of the endcap cone 100C can be greater than 10 cm, such as between 10-20 cm, between 10-30 cm, or longer. It can be preferrable in some cases to form longer endcap cones from a material that is more rigid, to prevent the endcap cone from bending as it is being directed through the skin, facia, and peritoneum.

Terms such as “top,” “bottom,” “side,” “front,” “back,” “right,” and “left” are not intended to provide an absolute frame of reference. Rather, the terms are relative and are intended to identify certain features in relation to each other, as the orientation of structures described herein can vary. The terms “comprising,” “including,” “having,” and the like are synonymous, are used in an open-ended fashion, and do not exclude additional elements, features, acts, operations, and so forth. Also, the term “or” is used in its inclusive sense, and not in its exclusive sense, so that when used, for example, to connect a list of elements, the term “or” means one, some, or all of the elements in the list.

Combinatorial language, such as “at least one of X, Y, and Z” or “at least one of X, Y, or Z,” unless indicated otherwise, is used in general to identify one, a combination of any two, or all three (or more if a larger group is identified) thereof, such as X and only X, Y and only Y, and Z and only Z, the combinations of X and Y, X and Z, and Y and Z, and all of X, Y, and Z. Such combinatorial language is not generally intended to, and unless specified does not, identify or require at least one of X, at least one of Y, and at least one of Z to be included. The terms “about” and “substantially,” unless otherwise defined herein to be associated with a particular range, percentage, or related metric of deviation, account for at least some manufacturing tolerances between a theoretical design and manufactured product or assembly, such as the geometric dimensioning and tolerancing criteria described in the American Society of Mechanical Engineers (ASME®) Y14.5 and the related International Organization for Standardization (ISO®) standards. Such manufacturing tolerances are still contemplated, as one of ordinary skill in the art would appreciate, although “about,” “substantially,” or related terms are not expressly referenced, even in connection with the use of theoretical terms, such as the geometric “perpendicular,” “orthogonal,” “vertex,” “collinear,” “coplanar,” and other terms.

The above-described embodiments of the present disclosure are merely examples of implementations to provide a clear understanding of the principles of the present disclosure. Many variations and modifications can be made to the above-described embodiments without departing substantially from the spirit and principles of the disclosure. In addition, components and features described with respect to one embodiment can be included in another embodiment. All such modifications and variations are intended to be included herein within the scope of this disclosure.

What is claimed is:

1. A surgical element carrier, comprising:
a housing dimensioned for insertion through a surgical incision during a surgery, the housing comprising:
a first housing portion;
a second housing portion attached and movable relative to the first housing portion between closed and open positions of the housing; and
a cover over the second housing portion, the cover forming a boundary of an interior space within the second housing portion, the cover comprising a plurality of slits that intersect with each other for a passageway through the cover, from outside to within the interior space within the second housing portion.

2. The surgical element carrier according to claim 1, wherein the plurality of slits comprises two intersecting slits that form a plus sign opening through the cover.

3. The surgical element carrier according to claim 1, wherein the plurality of slits form a star opening through the cover.

4. The surgical element carrier according to claim 1, wherein the plurality of slits comprises a plurality of intersecting perforations.

5. The surgical element carrier according to claim 1, further comprising a second plurality of slits that intersect with each other for a second passageway through the cover.

6. The surgical element carrier according to claim 1, further comprising a foam block secured to a top surface of the cover.

7. The surgical element carrier according to claim 1, further comprising:
a second plurality of slits that intersect with each other for a second passageway through the cover; and
a foam block secured to a top surface of the cover, the foam block positioned between the plurality of slits and the second plurality of slits.

8. The surgical element carrier according to claim 1, further comprising a magnetic member secured within the interior space within the second housing portion.

9. The surgical element carrier according to claim 1, further comprising at least one surgical element positioned in the first housing portion.

10. The surgical element carrier according to claim 1, further comprising a gel that occupies at least a portion of the interior space within the second housing portion.

11. The surgical element carrier according to claim 1, wherein the housing comprises a coned end at one end of the housing and an end tab at another end of the housing.

12. A surgical element carrier assembly, comprising:
a housing, comprising:
a first housing portion;
a second housing portion attached and movable relative to the first housing portion between closed and open positions of the housing; and
a cover over the second housing portion, the cover forming a boundary of an interior space within the second housing portion, the cover comprising a plurality of slits that intersect with each other for a passageway through the cover, from outside to within the interior space within the second housing portion; and
an endcap cone configured to fit over one end of the housing.

13. The surgical element carrier assembly according to claim 12, wherein the endcap cone is formed of rubber and can be expanded and pulled over an outer surface of the housing.

14. The surgical element carrier according to claim 12, wherein the plurality of slits comprises at least two intersecting slits.

15. The surgical element carrier assembly according to claim 12, wherein the plurality of slits comprises a plurality of intersecting perforations.

16. The surgical element carrier assembly according to claim 12, further comprising:
a second plurality of slits that intersect with each other for a second passageway through the cover; and
a foam block secured to a top surface of the cover, the foam block positioned between the plurality of slits and the second plurality of slits.

17. The surgical element carrier assembly according to claim 12, further comprising a magnetic member secured within the interior space within the second housing portion.

18. The surgical element carrier assembly according to claim 12, further comprising a gel that occupies at least a portion of the interior space within the second housing portion.

19. A surgical element carrier, comprising:
a housing, comprising:
a first housing portion;
a second housing portion attached and movable relative to the first housing portion between closed and open positions of the housing; and
a cover over the second housing portion, the cover forming a boundary of an interior space within the second housing portion, the cover comprising a plurality of slits that intersect with each other for a passageway through the cover, from outside to within the interior space within the second housing portion;
a magnetic member secured within the interior space within the second housing portion; and
at least one surgical element positioned in the first housing portion.

20. The surgical element carrier according to claim 19, further comprising:
a second plurality of slits that intersect with each other for a second passageway through the cover; and
a foam block secured to a top surface of the cover, the foam block positioned between the plurality of slits and the second plurality of slits.
